(19) United States
(12) Patent Application Publication
(10) Pub. No.: US 2004/0161803 A1
(43) Pub. Date: Aug. 19, 2004

(54) Methods and reagents for identifying weight loss promoters and therpeutic uses therefor

(76) Inventor: Barbara E. Corkey, Boston, MA (US)

Correspondence Address: Giulio A DeConti Jr, Lahive & Cockfield, 28 State Street, Boston, MA, 02109, US

(21) Appl. No.: 10/472,984

(22) PCT Filed: Apr. 1, 2002
(86) PCT No.: PCT/US02/10386

(51) Int. Cl. G01N 33/53; G01N 33/567; C12Q 1/26

(52) U.S. Cl. 435/7.2; 435/25

(57) ABSTRACT

Methods of identifying weight loss promoters are provided. Therapeutic methods utilizing compounds identified according to the methods of the invention are also provided.

### RELATED APPLICATIONS

This application claims the benefit of prior-filed provisional patent application U.S. Serial No. 60/280,682, filed Mar. 30, 2001. The entire contents of the above-referenced application is incorporated herein by this reference.

### GOVERNMENT RIGHTS

This invention was made at least in part with government support under grant: DK46200 awarded by the National Institutes of Health. The government has certain rights in this invention.

### BACKGROUND OF THE INVENTION

Based on data in the literature, the controlling step for free fatty acid (FFA) oxidation into CO₂ and partitioning, and esterification into complex lipids and triglycerides (TG), is at the level of the enzyme CPT-1. This enzyme has two isoforms, a muscle-type and a liver-type. During differentiation, preadipocytes express both isoforms. Mature fat cells express primarily the muscle isoform. Both isoforms are allosterically regulated by malonyl CoA but the muscle isoform is more sensitive. In aged animals, less FFA is oxidized and more stored. The reason for this is not known, but it could explain the increasing obesity with age.

Given the important role of CPT-1 in regulating FFA oxidation, there exists a need for understanding in greater detail the mechanism by which CPT-1 is regulated, for identifying modulators of CPT-1 activity which can promote fat oxidation (i.e. fat burning) and/or weight loss.

### SUMMARY OF THE INVENTION

The present invention is based, at least in part, on the discovery of a previously unrecognized role for carnitine palmitoyltransferase-1 (CPT-1). In particular, the present invention is based on the discovery that compounds which inhibit malonyl CoA binding to CPT-1 can promote CPT-1 activity and thereby promote free fatty acid (FFA) oxidation and weight loss. The interaction between CPT-1 and malonyl CoA is important in inhibiting CPT-1 activity, which is important in promoting FFA oxidation. Inhibition of the CPT-1/malonyl CoA interaction is proposed to regulate FFA oxidation and, consequently, weight loss.

The present inventors are the first to identify the interaction of CPT-1 with malonyl CoA as a target for identifying weight loss inhibitors.

In one embodiment, the invention provides methods for identifying a weight loss promoter comprising assaying the ability of a test compound to inhibit the interaction between CPT-1 and malonyl CoA to thereby inhibit the inhibition of CPT-1 activity and promote free fatty acid (FFA) oxidation. In a preferred embodiment, the methods are performed in a mitochondrion. In a preferred embodiment, the level of interaction of CPT-1 and malonyl CoA is measured by measuring the level of CoA-SH produced.

In another embodiment, the invention provides methods for identifying a weight loss promoter comprising assaying the ability of a test compound to inhibit the acetyl CoA carboxylase (ACC) catalyzed production of malonyl CoA. The ACC may be purified or in a cytosolic extract. In a preferred embodiment, the ability of a compound to inhibit malonyl CoA production is determined by measuring pH.

In a preferred embodiment, a weight loss promoter promotes FFA oxidation.

Other embodiments of the invention provide therapeutic methods for promoting FFA oxidation and/or weight loss by administering to a subject a weight loss promoter identified by one of the methods of the invention.

Other features and advantages of the invention will be apparent from the following detailed description and claims.

### DETAILED DESCRIPTION OF THE INVENTION

The present invention is based, at least in part, on the discovery of a previously unrecognized role for camitine palmitoyltransferase-1 (CPT-1). In particular, the present invention is based on the discovery that compounds which inhibit malonyl CoA binding to CPT-1 can promote CPT-1 activity and thereby promote free fatty acid (FFA) oxidation and weight loss. The interaction between CPT-1 and malonyl CoA is important in inhibiting CPT-1 activity, which is important in promoting FFA oxidation. Inhibition of the CPT-1/malonyl CoA interaction regulates FFA oxidation and, consequently, weight loss.

Accordingly, the present invention features methods of identifying weight loss modulators, in particular weight loss promoters.

Various aspects of the invention are described in further detail in the following subsections:

I. Screening Assays:

IA. Cell Free Assays

In one embodiment, an assay of the present invention is a cell-free assay in which a CPT-1 polypeptide, or biologically active portion thereof, is contacted with a test compound in the presence of malonyl CoA and the ability of the test compound to inhibit binding of malonyl CoA to the CPT-1 polypeptide or bioactive fragment thereof is determined. Binding of malonyl CoA to the CPT-1 polypeptide can be accomplished, for example, by coupling the CPT-1 polypeptide or malonyl CoA with a radioisotope or enzymatic label such that binding of malonyl CoA to the CPT-1 polypeptide can be determined, e.g., by detecting labeled malonyl CoA or polypeptide in a complex. For example, malonyl CoA or CPT-1 polypeptides can be labeled with ¹²⁵I, ³⁵S, ¹⁴C, or ³H, either directly or indirectly, and the radioisotope detected by direct counting of radioemission or by scintillation counting. Alternatively, substrates or polypeptides can be enzymatically labeled with, for example, horseradish peroxidase, alkaline phosphatase, or luciferase, and the enzymatic label detected by determination of conversion of an appropriate substrate to product.

Binding of malonyl CoA to the CPT-1 polypeptide can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In another embodiment, the assay includes contacting the CPT-1 polypeptide or biologically active portion thereof with malonyl CoA to form an assay mixture, contacting the assay mixture with a test compound, and determining the ability of the test compound to modulate binding between the CPT-1 polypeptide and malonyl CoA. In a preferred embodiment, the assay includes contacting the CPT-1 polypeptide or biologically active portion thereof with malonyl CoA to form an assay mixture, contacting the assay mixture with a test compound, and determining the ability of the test compound to interact with a CPT-1 polypeptide, wherein determining the ability of the test compound to interact with a CPT-1 polypeptide comprises determining the ability of the test compound to preferentially bind to CPT-1 or the bioactive portion thereof, for example, as compared to malonyl CoA.

In another embodiment, the assay is a cell-free assay in which a CPT-1 polypeptide or bioactive portion thereof is contacted with a test compound and the ability of the test compound to modulate (e.g., stimulate or inhibit) the activity of the CPT-1 polypeptide or biologically active portion thereof is determined. Preferably, determining the activity of the test compound to modulate the activity of CPT-1 is accomplished in the presence of malonyl CoA and the ability of CPT-1 to catalyze the following reaction:

long chain acyl CoA+carnitine→CoA-SH+acylcarnitine

is determined.

In one embodiment, the level of the products of the above reaction can be measured. For example, the level of CoA-SH can be measured by adding 5,5′-Dithiobis(2-nitrobenzoic acid) (DTNB) to the composition and determining the level of color as measured at 412 nm (Extinction Coefficient 13.6×10^{3cm-1}). In an alternative embodiment, determining the ability of the test compound to modulate the activity of a CPT-1 polypeptide can be accomplished, for example, by determining the ability of the CPT-1 polypeptide to modulate a downstream CPT-1 target molecule, e.g., an indirect assay for CPT-1 activity modulation. The term “CPT-1 target molecule” includes any non-CPT-1 molecule with which CPT-1 interacts, for example, in vitro or in vivo. Typically, CPT-1:“CPT-1 target molcule” interaction is a significant element of CPT-1 biological activity (e.g., enzymatic activity) or function (e.g., cellular function). Exemplary CPT-1 target molecules include, but are not limited to acyl CoA binding protein, acyl CoA synthase, fatty acid binding protein and CPT-1 products. In yet another embodiment, the cell-free assay involves contacting a CPT-1 polypeptide or biologically active portion thereof with a CPT-1 target molecule CPT-1 to form an assay mixture, contacting the assay mixture with a test compound, and determining the ability of the test compound to preferentially modulate the activity of a CPT-1 target molecule, as compared to the CPT-1.

In another embodiment of the present invention, the assay is a cell free assay for identifying a compound which can modulate the productionof malonyl CoA in which ACC polypeptide or a biologically active portion thereof is contacted with a test compound and the ability of the test compound to modulate (e.g., stimulate or inhibit) the activity of ACC (or a biologically active fragment thereof) is determined. Preferably, determining the ability of the test compound to modulate ACC is determined by detecting the following reaction, which is catalyzed by ACC:

Acetyl CoA+ATP+HCO₃⁻→Malonyl CoA+ADP+Pi+H⁺

In a preferred embodiment, a composition comprising ACC is contacted with a test compound in the presence of an ACC substrate (e.g., acetyl CoA), and readout of the reaction is measured by measuring the pH of the composition.

The above described assays may be performed using purified proteins or cytosolic extracts.

In more than one embodiment of the above assay methods of the present invention, it may be desirable to immobilize either CPT-1, malonyl CoA, a CPT-1 substrate or a CPT-1 target molecule or ACC to facilitate separation of complexed from uncomplexed forms of one or more components, as well as to accommodate automation of the assay. Binding of assay reagents, e.g., test compounds, malonyl CoA, CPT-1, CPT-1 substrates, CPT-1 target molecules, indicator reagents (e.g., fluorescent dyes, ACC and the like) can be accomplished in any vessel suitable for containing the reactants. Examples of such vessels include microtitre plates, test tubes, and micro-centrifuge tubes. In one embodiment, a fusion protein can be provided which adds a domain that allows protein reagents to be bound to a matrix. For example, glutathione-S-transferase/ACC fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound and appropriate reagents (or the test compound and either the non-adsorbed CPT-1 or ACC), and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of CPT-1 binding or activity or ACC binding or activity determined using standard techniques.

Other techniques for immobilizing proteins on matrices can also be used in the screening assays of the invention. For example, either a CPT-1 polypeptide or an ACC polypeptide can be immobilized utilizing conjugation of biotin and streptavidin. Biotinylated polypeptides can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with CPT-1 polypeptide or ACC polypeptides but which do not interfere with polypeptide binding or activity can be derivatized to the wells of the plate, and unbound polypeptide trapped in the wells by antibody conjugation. Methods for detecting such complexes, in addition to those described above for the GST-immobilized complexes, include immunodetection of complexes using antibodies reactive with the CPT-1 polypeptide or ACC polypeptide, as well as enzyme-linked assays which rely on detecting an enzymatic activity associated with the CPT-1 polypeptide or ACC polypeptide.

In yet another aspect of the invention, the CPT-1 polypeptides or ACC polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with CPT-1 or ACC (“binding proteins” or “target molecules”) and are involved in CPT-1 or ACC activity. Such target molecules are also likely to be involved in the regulation of cellular activities modulated by the CPT-1 or ACC polypeptides.

The two-hybrid system is based on the modular nature of most transcription factors, which consist of separable DNA-binding and activation domains. Briefly, the assay utilizes two different DNA constructs. In one construct, the gene that codes for a CPT-1 or ACC polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CPT-1- or ACC-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the CPT-1 or ACC polypeptide.

IB. Cell Based Assays

In one embodiment, an assay is a cell-based assay in which a cell which expresses a CPT-1 polypeptide, or biologically active portion thereof, is contacted with a test compound in the presence of malonyl CoA and/or CPT-1 substrates and the ability of the test compound to modulate the activity of the CPT-1 polypeptide, or biologically active portion thereof determined. The cell, for example, can be of mammalian origin or a yeast cell. The CPT-1 polypeptide, for example, can be expressed heterologously or native to the cell. In another embodiment, the assay is performed in a cellular organelle, e.g., a mitochondrion. In a preferred embodiment, an assay is a mitochondria-based assay in which a mitochondria comprising a CPT-1 polypeptide (e.g. having a CPT-1 polypeptide, or bioactive portion thereof, appropriately expressed the mitochondrial membrane) is contacted with a test compound in the presence of malonyl CoA and/or CPT-1 substrates and the ability of the test compound to modulate the activity of the CPT-1 polypeptide, or biologically active portion thereof determined. Determining the ability of the test compound to modulate the activity of a CPT-1 polypeptide, or biologically active portion thereof, can be accomplished by assaying for any of the activities of a CPT-1 polypeptide described herein. Determining the ability of the test compound to modulate the activity of a CPT-1 polypeptide, or biologically active portion thereof, can also be accomplished by assaying for the activity of a CPT-1 target molecule. In one embodiment, determining the ability of the test compound to modulate the activity of a CPT-1 polypeptide, or biologically active portion thereof, is accomplished by assaying for the ability of the test compound to preferentially bind CPT-1, e.g., as compared to malonyl CoA. In another embodiment, determining the ability of the test compound to modulate the activity of a CPT-1 polypeptide, or biologically active portion thereof, is accomplished by assaying for production of CoA-SH as described above. In a preferred embodiment, the cell or the mitochondrion which expresses the CPT-1 polypeptide, or biologically active portion thereof, is contacted with the test compound in the presence of malonyl CoA. In yet another preferred embodiment, the cell or mitochondria is contacted with a compound which stimulates or inhibits a CPT-1-associated activity (e.g., FFA oxidation) and the ability of a test compound to modulate the CPT-1-associated activity is determined.

In another embodiment, an assay is a cell-based assay in which a cell which expresses a CPT-1 polypeptide, or biologically active portion thereof, is contacted with a bioactive peptide derived from a CPT-1 target molecule and a test compound and the ability of the test compound to modulate the activity of the CPT-1 polypeptide, or biologically active portion thereof, determined. In yet another embodiment, a bioactive peptide for use in the methodology of the instant derived from the amino acid sequence of ACC. In another embodiment, the bioactive peptide corresponds to a bioactive domain of ACC (e.g., a CPT-1 interacting domain). In yet another embodiment, the bioactive peptide corresponds to a trafficking motif of ACC.

Compounds identified in the screening assays of the invention as weight loss promoters can be further tested for the ability to modulate (e.g., promote) FFA oxidation in adipocytes or animals (e.g., animal models for obesity) using standard methods known to those skilled in the art.

II. Assay reagents

IIA. Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404406); (Cwirla etal. (1990) Proc. Natl. Acad. Sci. 87:6378-6382); (Felici (1991) J. Mol. Biol. 222:301-310); (Ladner supra.).

In a preferred embodiment, the library is a natural product library.

IIB. Antibodies, Bioactive Fragments and Fusion Proteins

Another aspect of the invention features biologically active portions (i.e., bioactive fragments) of CPT-1 or ACC, including polypeptide fragments suitable for use as immunogens to raise anti-CPT-1 antibodies or ACC antibodies or to make CPT-1 or ACC fusion proteins. In one embodiment, CPT-1 or ACC immunogens or bioactive fragments can be generated from CPT-1 or ACC isolated from cells or tissue sources by an appropriate purification scheme using standard protein purification techniques. In another embodiment, CPT-1 or ACC immunogens or bioactive fragments are produced by recombinant DNA techniques. Alternative to recombinant expression, a CPT-1 or ACC immunogens or bioactive fragments can be synthesized chemically using standard peptide synthesis techniques.

An immunogen, bioactive fragment or fusion protein, as used herein is preferably “isolated” or “purified”. The terrns “isolated” and “purified” are used interchangeably herein. “Isolated” or “purified” means that the immunogen, bioactive fragment or fusion protein is substantially free of cellular material or other contaminating proteins from the cell or tissue source from which the polypeptide is derived, substantially free of other protein fragments, for example, non-desired fragments in a digestion mixture, or substantially free from chemical precursors or other chemicals when chemically synthesized. The language “substantially free of cellular material” includes preparations in which the polypeptide is separated from other components of the cells from which it is isolated or recombinantly produced. In one embodiment, the language “substantially free of cellular material” includes preparations of CPT-1 polypeptide having less than about 30% (by dry weight) of non-CPT-1 polypeptide (also referred to herein as a “contaminating protein”), more preferably less than about 20% of non-CPT-1 polypeptide, still more preferably less than about 10% of non-CPT-1 polypeptide, and most preferably less than about 5% non-CPT-1 polypeptide. In another embodiment, the language “substantially free of cellular material” includes preparations of ACC polypeptide having less than about 30% (by dry weight) of non-ACC polyepptide (also referred to herein as a “contaminating protein”), more preferably less than about 20% of non-ACC polypeptide, still more preferably less than about 10% of non-ACC polypeptide, and most preferably less than about 5% non-ACC polypeptide. When the immunogen, bioactive portion or fusion protein is recombinantly produced, it is also preferably substantially free of culture medium, i.e., culture medium represents less than about 20%, more preferably less than about 10%, and most preferably less than about 5% of the volume of the polypeptide preparation. When the immunogen, bioactive fragment or fusion protein is produced by, for example, chemical or enzymatic processing from isolated or purified CPT-1 or ACC protein, the preparation is preferably free of enzyme reaction components or chemical reaction components and is free of non-desired CPT-1 or ACC fragments, i.e., the desired polypeptide represents at least 75% (by dry weight) of the preparation, preferably at least 80%, more preferably at least 85%, and even more preferably at least 90%, 95%, 99% or more or the preparation.

The language “substantially free of chemical precursors or other chemicals” includes preparations of polypeptide in which the polypeptide is separated from chemical precursors or other chemicals which are involved in the synthesis of the polypeptide. In one embodiment, the language “substantially free of chemical precursors or other chemicals” includes preparations having less than about 30% (by dry weight) of chemical precursors or reagents, more preferably less than about 20% chemical precursors or reagents, still more preferably less than about 10% chemical precursors or reagents, and most preferably less than about 5% chemical precursors or reagents.

Bioactive fragments of CPT-1 or ACC include polypeptides comprising amino acid sequences sufficiently identical to or derived from the amino acid sequence of the CPT-1 protein or the ACC protein, respectively, which include less amino acids than the full length protein, and exhibit at least one biological activity of the full-length protein. Typically, biologically active portions comprise a domain or motif with at least one activity of the fill-length protein. A biologically active portion of a CPT-1 or ACC can be a polypeptide which is, for example, 50, 100, 150, 200, 250, 300, 350, 400, 450, 500, 550, 600, 650, 700, 750, 800, 850, 900, 950, 1000 or more amino acids in length. For example, in one embodiment, a bioactive portion of a CPT-1 protein or ACC protein comprises at least a catalytic domain. In a preferred embodiment, a bioactive portion of a CPT-1 protein comprises at least a transferase domain domain. In another preferred embodiment, a bioactive portion of an ACC protein comprises at least a carboxyl transferase domain. A preferred activity of a catalytic domain is an enzymatic activity possessed by the full length CPT-1 or ACC protein. Additional preferred CPT-1 domains include, but are not limited to, a cytoplasmic domain, transmembrane or mitochindrial intermenbrane domain and a mitochondrial domain.

Moreover, other biologically active portions, in which other regions of the protein are deleted, can be prepared by recombinant techniques and evaluated for one or more of the functional activities of a native CPT-1 or ACC protein.

To determine the percent identity of two amino acid sequences (or of two nucleotide or amino acid sequences), the sequences are aligned for optimal comparison purposes (e.g., gaps can be introduced in the first sequence or second sequence for optimal alignment). The amino acid residues or nucleotides at corresponding amino acid positions or nucleotide positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. BLAST alignments can be generated and percent identity calculated using BLAST protein searches (e.g., the XBLAST program) using CPT-1, ACC or a portion thereof as a query, score=50, wordlength=3.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Research 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

The invention also provides CPT-1 and ACC chimeric or fusion proteins. As used herein, a CPT-1 or ACC “chimeric protein” or “fusion protein” comprises a CPT-1 or ACC polypeptide operatively linked to a non-CPT-1 polypeptide or non-ACC polypeptide, respectively. A “CPT-1 polypeptide” or “ACC polypeptide” refers to a polypeptide having an amino acid sequence corresponding to the CPT-1 or ACC protein, respectively, whereas a “non-CPT-1 polypeptide” or “non-ACC polypeptide” refers to a polypeptide having an amino acid sequence corresponding to a protein which is not substantially identical to the CPT-1 protein or ACC protein. Within a fusion protein the CPT-1 or ACC polypeptide can correspond to all or a portion of a CPT-1 or ACC protein. In a preferred embodiment, a CPT-1 or ACC fusion protein comprises at least one biologically active portion of a CPT-1 or ACC protein, respectively. In another preferred embodiment, a CPT-1 or ACC fusion protein comprises at least two biologically active portions of a CPT-1 or ACC protein, respectively. Within the fusion protein, the term “operatively linked” is intended to indicate that the CPT-1 or ACC polypeptide and the non-CPT-1 polypeptide or non-ACC polypeptide are fused in-frame to each other. The non-CPT-1 polypeptide or non-ACC polypeptide can be fused to the N-terminus or C-terminus of the CPT-1 polypeptide or ACC polypeptide, respectively.

For example, in one embodiment, the fusion protein is a GST-fusion protein in which the CPT-1 or ACC sequences are fused to the C-terminus of the GST sequences. In another embodiment, the fusion protein is a chitin fusion protein in which the CPT-1 or ACC sequences are fused to the N-terminus of chitin sequences. Such fusion proteins can facilitate the purification of recombinant CPT-1 or ACC.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety. A CPT-1- or ACC-encoding nucleic acid can be cloned into such an expression vector such that the fusion moiety is linked in-frame to the CPT-1 or ACC polypeptide when expressed.

A CPT-1 polypeptide or ACC polypeptide, or a portion or fragment of CPT-1 or ACC, can also be used as an immunogen to generate antibodies that bind CPT-1 or ACC or that block CPT-1/ACC binding using standard techniques for polyclonal and monoclonal antibody preparation. A full-length polypeptide can be used or, alternatively, the invention provides antigenic peptide fragments for use as immunogens. Preferably, an antigenic fragment comprises at least 8 amino acid residues of the amino acid sequence of CPT-1 (as set forth in GenBank Accession no. P50416) or ACC (e.g., ACC1 as set forth in GenBank Accession no. ACC501139 or ACC2 as set forth in GenBank Accession no. AAB58382) and encompasses an epitope of CPT-1 or ACC such that an antibody raised against the peptide forms a specific immune complex with CPT-1 or ACC, respectively. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of CPT-1 or ACC that are located on the surface of the protein, e.g., hydrophilic regions.

A CPT-1 or ACC immunogen typically is used to prepare antibodies by immunizing a suitable subject, (e.g., rabbit, goat, mouse or other mammal) with the immunogen. An appropriate immunogenic preparation can contain, for example, recombinantly expressed CPT-1 or ACC polypeptide or a chemically synthesized CPT-1 or ACC polypeptide. The preparation can further include an adjuvant, such as Freund's complete or incomplete adjuvant, or similar immunostimulatory agent. Immunization of a suitable subject with an immunogenic CPT-1 or ACC preparation induces a polyclonal anti-CPT-1 or anti-ACC antibody response, respectively.

Accordingly, another aspect of the invention pertains to anti-CPT-1 or anti-ACC antibodies. The term “antibody” as used herein refers to immunoglobulin molecules and immunologically active portions of immunoglobulin molecules, i.e:, molecules that contain an antigen binding site which specifically binds (immunoreacts with) an antigen, such as CPT-1 or ACC. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind CPT-1 or ACC. The term “monoclonal antibody” or “monoclonal antibody composition”, as used herein, refers to a population of antibody molecules that contain only one species of an antigen binding site capable of immunoreacting with a particular epitope of CPT-1 or ACC. A monoclonal antibody composition thus typically displays a single binding affinity for a particular CPT-1 or ACC polypeptide with which it immunoreacts.

Polyclonal anti-CPT-1 or anti-ACC antibodies can be prepared as described above by immunizing a suitable subject with a CPT-1 or ACC immunogen, respectively. The antibody titer in the immunized subject can be monitored over time by standard techniques, such as with an enzyme linked immunosorbent assay (ELISA) using immobilized CPT-1 or ACC. If desired, the antibody molecules can be isolated from the mammal (e.g., from the blood) and further purified by well known techniques, such as protein A chromatography to obtain the IgG fraction. At an appropriate time after immunization, e.g., when the anti-CPT-1 or anti-ACC antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:53946; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med, 54:387402; M. L. Gefter et al. (1977) Somatic Cell Genet. 3:231-36). Briefly, an immortal cell line (typically a myeloma) is fused to lymphocytes (typically splenocytes) from a mammal immunized with a CPT-1 or ACC immunogen as described above, and the culture supernatants of the resulting hybridoma cells are screened to identify a hybridoma producing a monoclonal antibody that binds CPT-1 or ACC, respectively.

Any of the many well known protocols used for fusing lymphocytes and immortalized cell lines can be applied for the purpose of generating an anti-CPT-1 or anti-ACC monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind CPT-1 or ACC, e.g., using a standard ELISA assay.

Alternative to preparing monoclonal antibody-secreting hybridomas, a monoclonal anti-CPT-1 or anti-ACC antibody can be identified and isolated by screening a recombinant combinatorial immunoglobulin library (e.g., an antibody phage display library) with CPT-1 or ACC to thereby isolate immunoglobulin library members that bind CPT-1 or ACC, respectively. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrard et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

An anti-CPT-1 or anti-ACC antibody (e.g., monoclonal antibody) can be used to isolate CPT-1 or ACC, bioactive portions thereof, or fusion proteins by standard techniques, such as affinity chromatography or immunoprecipitation. Detection of anti CPT-1 or anti-ACC antibodies can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

IIC. Recombinant Expression Vectors and Assay Cells

Another aspect of the invention pertains to vectors, preferably expression vectors, for producing fusion protein reagents of the instant invention. As used herein, the term “vector” refers to a nucleic acid molecule capable of transporting another nucleic acid to which it has been linked. A preferred vector is a “plasmid”, which refers to a circular double stranded DNA loop into which additional DNA segments can be ligated. In the present specification, “plasmid” and “vector” can be used interchangeably as the plasmid is the most commonly used form of vector.

The recombinant expression vectors of the invention comprise a nucleic acid that encodes, for example CPT-1 or ACC or a bioactive fragment of CPT-1 or ACC, in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). The expression vectors can be introduced into host cells to thereby produce proteins, including fusion proteins or peptides.

The recombinant expression vectors of the invention can be designed for expression of CPT-1 or ACC polypeptides in prokaryotic or eukaryotic cells. For example, CPT-1 or ACC polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990).

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by. acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Purified fusion proteins are particularly useful in the cell-free assay methodologies of the present invention.

In yet another embodiment, a CPT-1 or ACC-encoding nucleic acid is expressed in mammalian cells, for example, for use in the cell-based assays described herein. When used in mammalian cells, the expression vector's control functions are often provided by viral regulatory elements. In another embodiment, the recombinant mammalian expression vector is capable of directing expression of the nucleic acid preferentially in a particular cell type (e.g., tissue-specific regulatory elements are used to express the nucleic acid).

Another aspect of the invention pertains to assay cells into which a recombinant expression vector has been introduced. An assay cell can be prokaryotic or eukaryotic, but preferably is eukaryotic. Vector DNA can be introduced into prokaryotic or eukaryotic cells via conventional transformation or transfection techniques. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

An assay cell of the invention, can be contacted with a test compound and assayed for any CPT-1 and/or ACC biological activity (by methods described herein or as known in the art) in order to identify the compound as an weight loss modulator.

III. Pharmaceutical Compositions

This invention further pertains to weight loss modulators identified by the above-described screening assays. Weight loss modulators identified by the above-described screening assays can be tested in an appropriate animal model. For example, an weight loss modulator identified as described herein can be used in an animal model to determine the efficacy, toxicity, or side effects of treatment with such a modulator. Alternatively, a modulator identified as described herein can be used in an animal model to determine the mechanism of action of such an agent. Furthermore, this invention pertains to uses of weight loss modulators identified by the above-described screening assays for therapeutic treatments as described infra.

Accordingly, the weight loss modulators of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradernal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdernal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the EDS0 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

IV Methods of Treatment

The present invention further provides for therapeutic methods of treating a subject having an weight disorder, for example, obesity, overweight, reduced insulin sensitivity, insulin resistance, diabetes (e.g., Type II diabetes), cachexia, or anorexia. The therapeutic methods a particularly useful for treating obese subjects and/or obese diabetics.

A preferred aspect of the invention pertains to methods of modulating CPT-1/substrate (e.g, CPT-1/malonyl CoA) interactions for therapeutic purposes. Accordingly, in an exemplary embodiment, the therapeutic method of the invention involves.

The effectiveness of treatment of a subject with an weight loss modulator can be accomplished by (i) detecting the level of FFA oxidation, blood insulin levels or, alternatively, body weight in the subject prior to treating with an appropriate modulator; (ii) detecting the level of FFA oxidation, blood insulin or, alternatively, body weight in the subject post treatment with the modulator; (iii) comparing the levels pre-administration and post administration; and (iv) altering the administration of the modulator to the subject accordingly. Increased administration of the modulator may be desirable if the subject continues to demonstrate overweight or obesity, or reduced FFA oxidation. Obese subjects, for example, typically exhibit normal blood glucose, high insulin levels and increased levels of blood FFA. Effective treatment is evidenced by decreased blood FFA (due to increased FFA oxidation). Decreased levels of blood insulin are also indicative of effective treatment (due, for example, to increased insulin sensitivity). The pre- and post-treatment profiles of an obese-diabetic are similar, except that they typically exhibit high blood glucose levels, as well, when detection is performed pre-treatment. Decreased blood glucose is further indicative of effective treatment in these subjects.

This invention is further illustrated by the following examples which should not be construed as limiting. The contents of all references, patents and published patent applications cited throughout this application are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a weight loss promoter, comprising contacting a composition comprising CPT-1 or a bioactive fragment thereof with a test compound in the presence of malonyl CoA and determining the ability of the test compound to stimulate binding of the CPT-1 or bioactive fragment to malonyl CoA, such that a compound which is a weight loss promoter is identified.

2. A method for identifying a weight loss promoter, comprising contacting a composition comprising CPT-1 or a bioactive fragment thereof with a test compound in the presence of malonyl CoA and CPT-1 substrates and determining the ability of the test compound to inhibit activity of the CPT-1 or bioactive fragment, such that a compound which is a weight loss promoter is identified.

3. A method for identifying a weight loss promoter, comprising contacting a mitochondria comprising CPT-1 or a bioactive fragment thereof with a test compound in the presence of malonyl CoA and determining the ability of the test compound to inhibit binding of the CPT-1 or bioactive fragment to malonyl CoA, such that a compound which is a weight loss promoter is identified.

4. A method for identifying a weight loss promoter, comprising contacting a mitochondria comprising CPT-1 or a bioactive fragment thereof with a test compound in the presence of malonyl CoA and CPT-1 substrates and determining the ability of the test compound to stimulate activity of the CPT-1 or bioactive fragment, such that a compound which is a weight loss promoter is identified.

5. The method of claim 2 or 4, wherein detecting the ability of the test compound to modulate activity of the CPT-1 or bioactive fragment comprises detecting a change in the level of CoA-SH.

6. The method of claim 5, wherein the composition further comprises 5,5′-Dithiobis(2-nitrobenzoic acid) (DTNB).

7. The method of claim 6, wherein detecting a change in the level of CoA-SH comprises detecting a change in the amount of color detected at 412 nm (Extinction Coefficient 13.6×10^{3cm-1}).

8. The method of claim 2 or 4, wherein inhibition of CPT-1 activity by malonyl CoA is relieved.

9. The method of claim 2 or 4, wherein free fatty acid oxidation is increased.

10. A method for identifying a weight loss promoter, comprising contacting a composition comprising ACC or a bioactive fragment thereof with a test compound in the presence of an ACC substrate and determining the ability of the test compound to inhibit production of malonyl CoA, such that a compound which is a weight loss promoter is identified.

11. The method of claim 10, wherein the ACC substrate is Acetyl CoA.

12. The method of claim 10, wherein determining the ability of the test compound to modulate production of malonyl CoA comprises determining the pH of the composition.

13. The method of claim 10, wherein the composition comprises purified ACC or bioactive fragment.

14. The method of claim 10, wherein the composition is a cytosolic extract.

15. The method of claim 11, wherein malonyl CoA production is decreased.

16. The method of claim 11, wherein free fatty acid oxidation is increased.

17. A method of promoting free fatty acid oxidation in a subject comprising administering to said subject a weight loss promoter identified according to the method of any one of claims 1-16, such that free fatty acid oxidation in said subject is promoted.

18. A method of weight loss in a subject, comprising administering to said subject a weight loss promoter identified according to the method of any one of claims 1-16, such that weight loss in said subject is enhanced.
